(19) United States
(12) Patent Application Publication
(10) Pub. No.: US 2017/0156317 A1
(43) Pub. Date: Jun. 8, 2017

(54) METHODS AND APPARATUS FOR PASSIVE REDUCTION OF NOSOCOMIAL INFECTIONS IN CLINICAL SETTINGS, AND FABRICS, YARNS, AND FILAMENTS FOR USE IN CONNECTION THEREWITH

(71) Applicant: PHILADELPHIA UNIVERSITY, Philadelphia, PA (US)

(72) Inventors: Alexander A. Messinger, Ardmore, PA (US); Diana R. Cundell, Philadelphia, PA (US); Brian R. George, Pottstown, PA (US)

(21) Appl. No.: 15/186,221

(22) Filed: Jun. 17, 2016

Related U.S. Application Data
(63) Continuation of application No. 14/944,307, filed on Nov. 18, 2015, now abandoned.
Application No. 14/944,307 is a continuation of application No. 13/112,252, filed on May 20, 2011, now abandoned.
Application No. 13/112,252 is a continuation-in-part of application No. 13/052,592, filed on Mar. 21, 2011, now abandoned.
Application No. 13/052,592 is a continuation of application No. 12/705,843, filed on Feb. 15, 2010, now abandoned.
(60) Provisional application No. 61/351,390, filed on Jun. 4, 2010.

(51) Int. Cl. A01N 25/34; A41D 31/00; A61L 9/16; A41D 13/12; A01N 31/16; D06M 13/152

(52) U.S. Cl. CPC A01N 25/34; A01N 31/16; D06M 13/152; A61L 9/16; A41D 13/12; A41D 31/0077; A61L 2209/15; A61L 2209/21; A41D 2400/34

(57) ABSTRACT

A method for passively reducing nosocomial infections by providing fabrics for patient contact only as media for air filtration in areas with patient populations, which fabrics have been treated with a solution of eugenol of sufficient strength and for sufficient time to reduce the percentage of viable microbes in the fabrics by at least 2 log units.

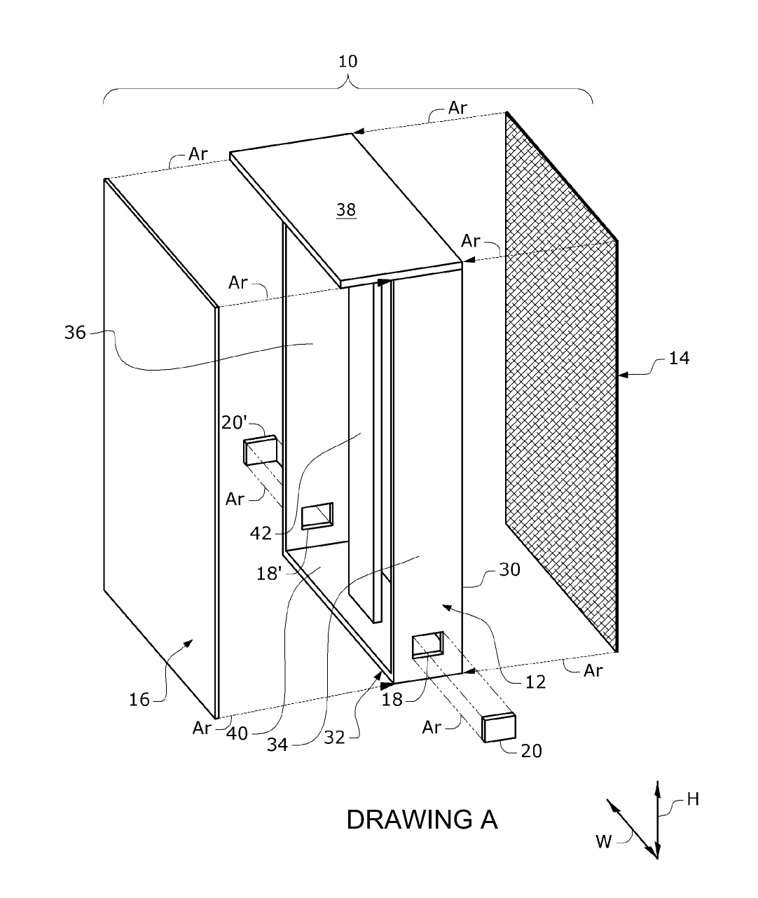

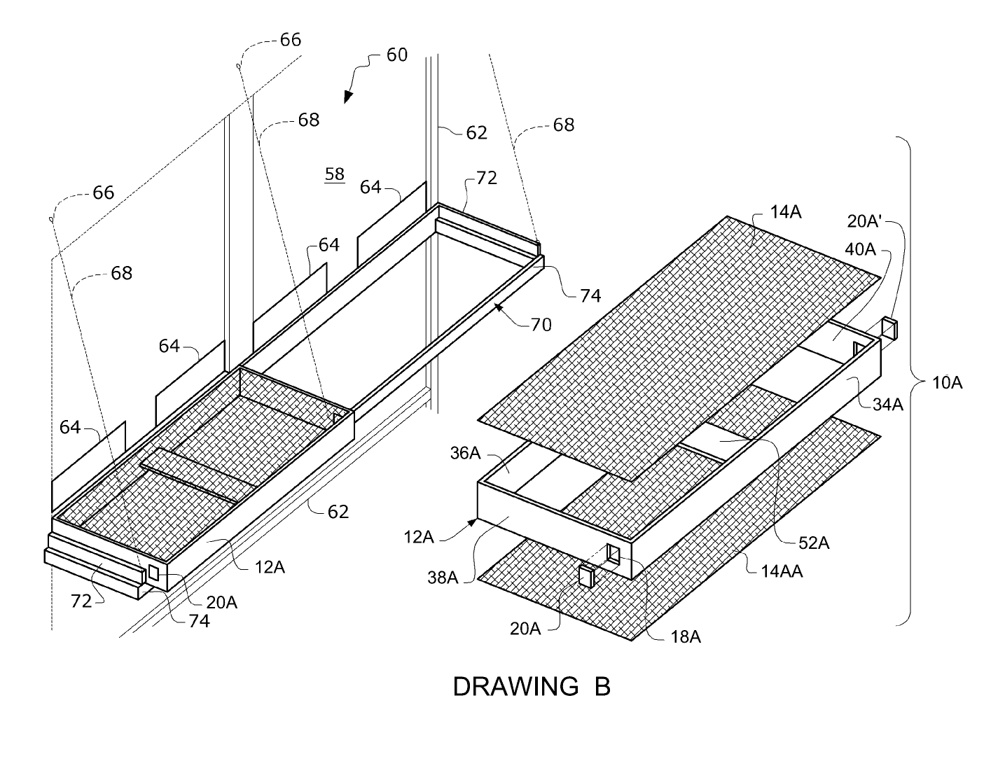

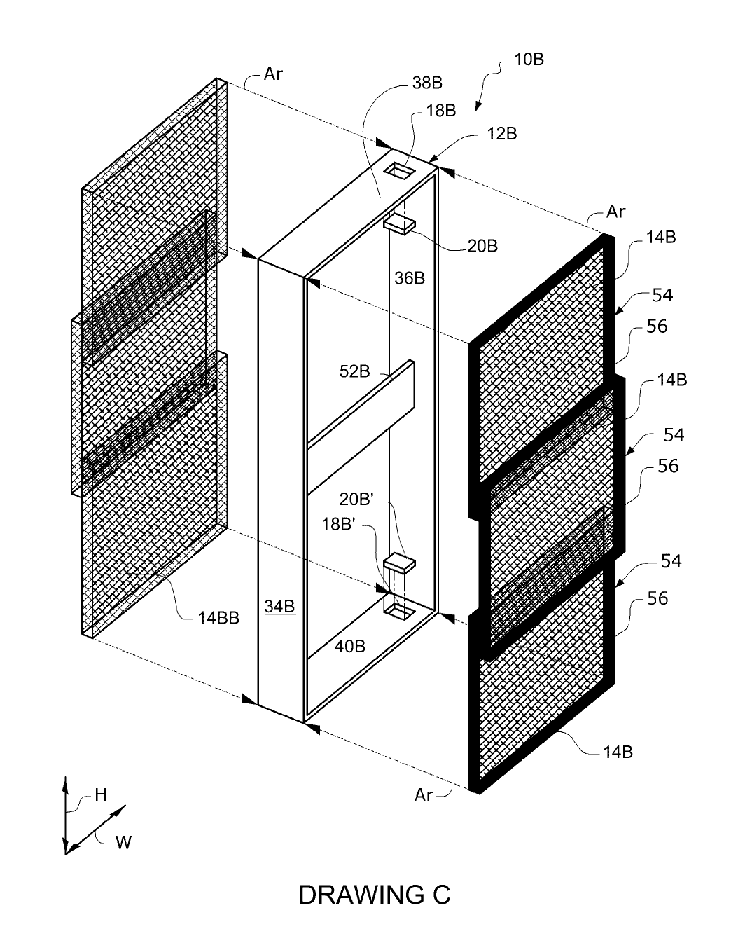

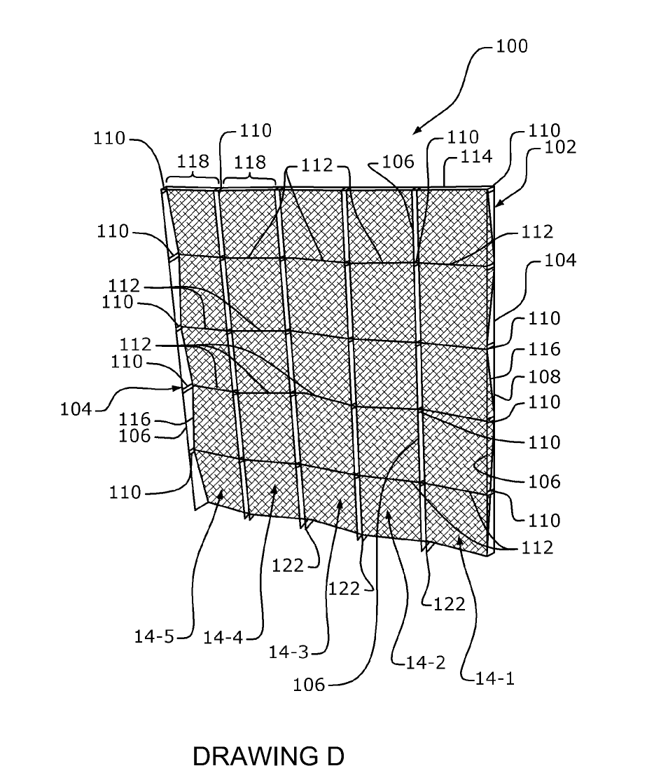

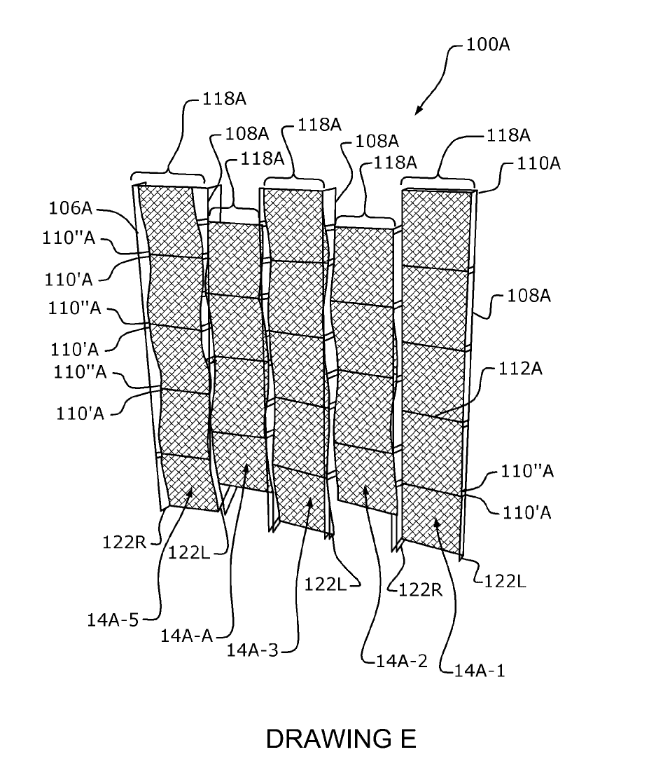

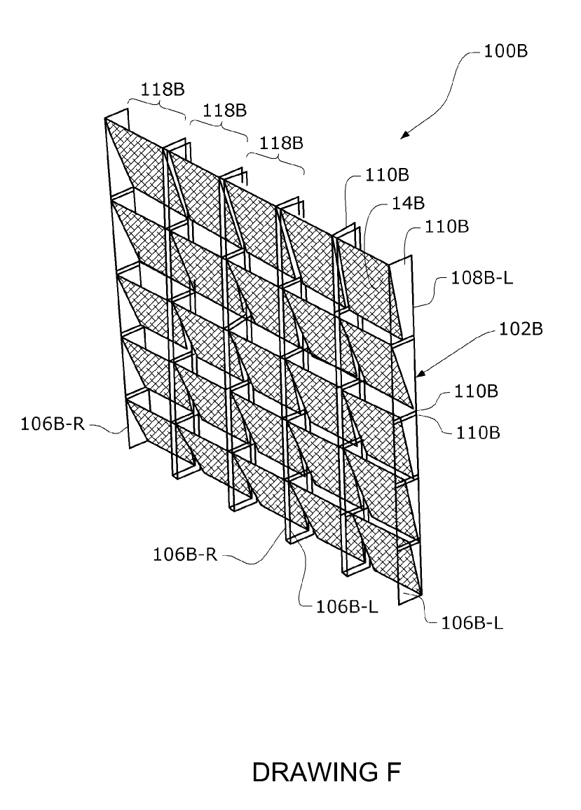

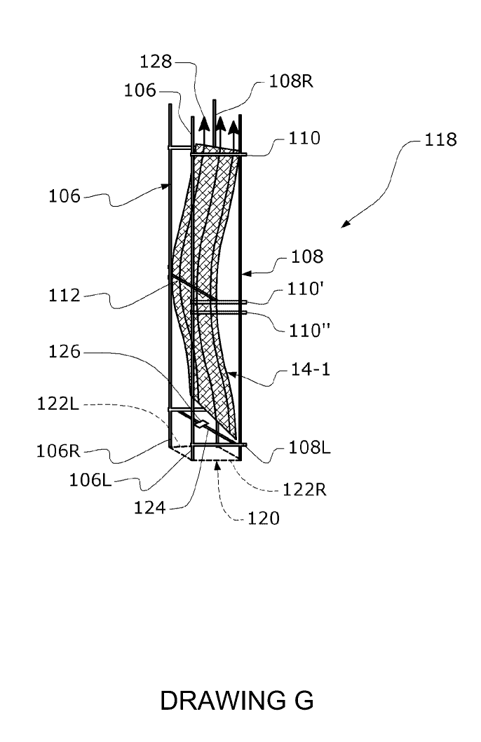

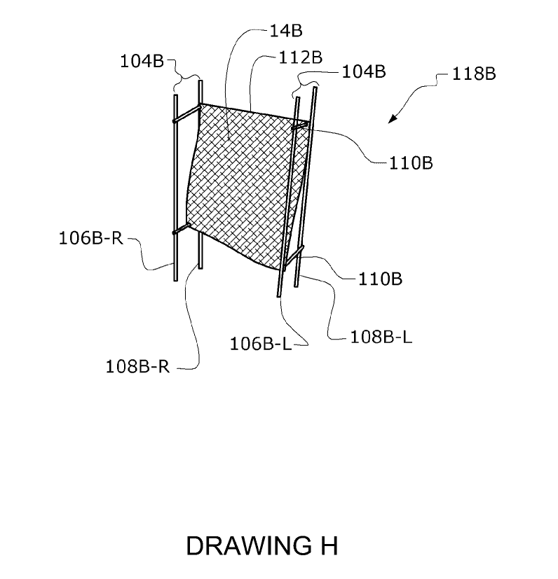

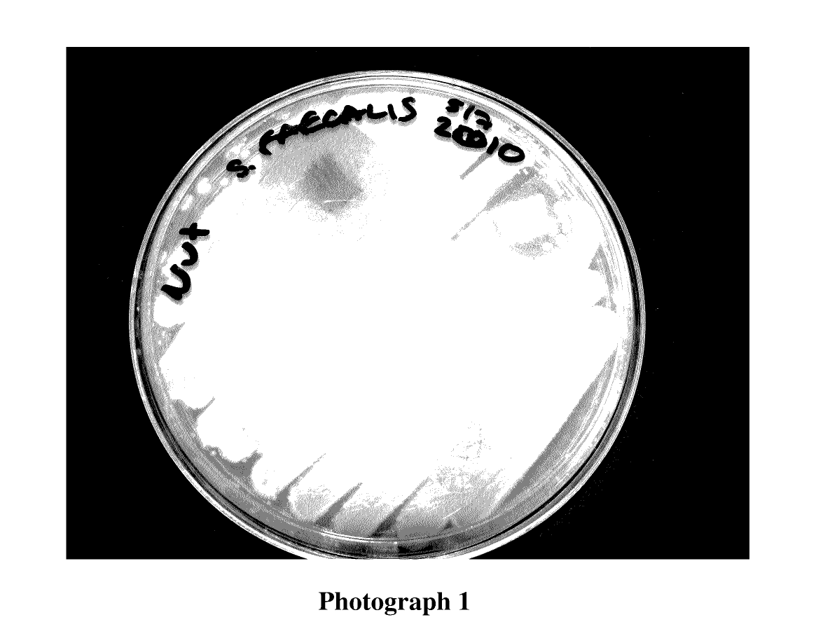

### CROSS-REFERENCE TO RELATED PATENT APPLICATIONS

This patent application is a 35 USC 120 continuation application of pending U.S. utility patent application Ser. No. 14/944,307 entitled “Methods and Apparatus for Passive Reduction of Nosocomial Infections in Clinical Settings, and Fabrics, Yarns, and Filament for Use in Connection Therewith, filed 18 Nov. 2015, which is a continuation application of U.S. utility patent application Ser. No. 13/112,252 filed 20 May 2011, which claims the benefit of the priority of U.S. provisional application Ser. No. 61/351,390, filed 4 Jun. 2010 in the names of Alexander A. Messinger, Diana R. Lundell and Brian R. George, under 35 USC 119 and which is a 35 USC 120 continuation-in-part of pending U.S. utility patent application Ser. No. 12/705,843 entitled “Methods and Apparatus for Combating Sick Building Syndrome”, filed 15 Feb. 2010 in the names of the aforementioned Alexander A. Messinger, Diana R. Lundell and Brian R. George, and is also a 35 USC 120 continuation-in-part of pending U.S. utility patent application Ser. No. 13/052,592, entitled “Methods for Imparting Anti-Microbial, Microbiocidal Properties to Fabrics, Yarns and Filaments, and Fabrics, Yarns and Filaments Embodying Such Properties”, filed 21 Mar. 2011 in the names of the aforementioned Alexander A. Messinger, Diana R. Lundell, and Brian R. George, and in the names of Bhalchandra Dhamankar and Ekaterina Shumilova.

### BACKGROUND OF INVENTION—FIELD OF THE INVENTION

This invention relates to methods, apparatus and fabrics for passively reducing nosocomial infections, especially in clinical settings, and to fabrics, yarns and filaments for use in connection with such methods and apparatus.

### BACKGROUND OF THE INVENTION—DESCRIPTION OF THE NOSOCOMIAL INFECTION

### Problem and the Prior Art

Nosocomial infections have been monitored and various strategies implemented to try and manage them since 1985 (http://www.cdc.gov/ncidod/eid/vol4no3/weinstein.htm) However, despite increased surveillance, awareness and attention to hospital cleanliness, the Center for Disease Control continues to report an incidence of between 5 and 6 nosocomial infections for every 1,000 hospital admissions. Nosocomial disease is now the fourth leading cause of death in the United States (http://www.cdc.gov/ncidod/eid/vol4no3/weinstein.htm).

These infections are troublesome in several respects. First, the majority of them are not vaccine-preventable. Vaccines are unlikely to be developed against them in the near future.

Second, the infectious microbes are not destroyed using conventional cleaning methods. Hospitals have germicidal lamps and specialized soaps and floor cleansers. In spite of this bacteria, particularly Pseudomonas aeruginosa, methicillin-resistant Staphylococcus aureus (MRSA) and Escherichia coli, are able to survive and spread among patients. These bacteria produce the greatest incidence of morbidity and mortality in hospitals (http://www.cdc.gov/ncidod/eid/vol4no3/weinstein.htm).

Third, the majority of these recalcitrant bacteria are also antibiotic resistant. In a 2001 survey of 87 New Jersey hospitals, three genres of resistant bacteria were identified as being the most dangerous: MRSA, vancomycin-resistant Enterococci (VRE), and gram negative enteric bacilli (including Klebsiella pneumoniae, E. coli and Enterococci).

Recent studies have also added a fourth antibiotic-resistant bacteria to the group. Studies now suggest that nosocomial Clostridium difficile infections are now 25% more frequent than MRSA infections. This is a troublesome situation as the bacterium Clostridium difficile is resistant, also, to alcohol-based hand sanitizers and produces antibiotic-resistant spores able to survive in the environment for several months (http://www.medicinenet.com/script/main/art.asp?articlekey=114613).

Much of the spread of these bacteria is by passive transfer involving the scrubs, gowns and white coats of hospital personnel; this accelerates the movement of infectious microbes through hospital settings. In spite of rigorous barrier maintenance, including hand washing, laundering and changing of these items, patients in intensive care units, who are usually the weakest patients, are between 5 and 10 times more likely than patients in other parts of a hospital to contract one of these antibiotic-resistant bacteria and either to require extended hospital stays or to die from the infection (http://chestjournal.chestpubs.org/content/115/suppl_1/34S.full).

Current design of many hospitals, clinics and other health care facility buildings seeks to maximize energy efficiency and comfort for the inhabitants using centralized heating and cooling systems. Combined with the use of inexpensive building materials such as particle board, drywall and acoustical tile, the modern design and construction approach has fostered environments in which nosocomial infections have been increasingly prevalent and difficult to treat.

Products currently promoted to remove airborne contaminants primarily focus on allergens and trap them in electrostatically-charged filters, which require periodic replacement or cleaning.

Silver has proven useful acting as a molecular poison against a broad spectrum of bacteria. Chitin, sometimes called “chitosan”, is also used as an antibacterial. It is easy to obtain and more environmentally friendly than heavy metals such as silver. Triclosan is another commonly used substance, and is the active ingredient in antibacterial hand washes, toothpastes and the like.

These materials all have disadvantages, one of the greatest of which is cost. Especially in the case of silver, the current cost is about $6.00 per ounce, and is predicted to rise to as high as $25.00 per ounce or greater within the next several years. Also, toxicity is a problem. Triclosan may break down in water to produce chloroform and dioxins.

Clove oil is a known antibacterial effective against staphylococcus aureus, pseudomonas aeruginosa, clostridium perfringens and Escherichia coli, and is an antifungal effective against candida, apergillus, penicillium and trychophyton.

Clove oil is currently used in mouth care products for toothaches and as a breath freshener, as a filling or cement material as zinc oxide eugenol for tooth repair, as rose oil in perfumery and soaps, as an antioxidant for plastic and rubber, as an insecticide, and for sanitation purposes.

Unfortunately, none of these approaches or materials have proven to be successful in removing or abrogating nosocomial infections in clinical settings.

### SUMMARY OF THE INVENTION

In one of its aspects, this invention provides fabrics treated to inhibit environmental isolates of S. aureus, which on antibiotic testing proved to be multi-resistant, and to inhibit a spore-bearing microbe Bacillus cereu. In vitro testing, using clinical strains of six gram positive bacteria including S. faecalis (Enterobacter faecalis), VRE, Mycobacterium smegmatis (used as an analog and tuberculosis), Bacillus cereus (used as an analog of anthrax) and two strains of streptococci (S. pneumoniae and S. agalacticae), with fabrics treated in accordance with the invention, show significant antimicrobial activity respecting all four strains.

These results are particularly important given 34% of all nosocomial infections can be attributed to gram positive bacteria (http://www.cdc.gov/ncidod/eid/vol4no3/weinstein.htm). When fabrics treated in accordance with the invention were evaluated with one strain of the gram negative bacterium P. aeruginosa, favorable results were evident.

Current approaches have not been successful in removing/abrogating the issue of nosocomial infection. Hence, the characteristic of fabrics treated in accordance with the invention to inhibit environmental isolates of S. aureus, which on antibiotic testing proved to be multi-resistant, and to inhibit a spore-bearing microbe Bacillus cereu, is important and valuable. In vitro testing using clinical strains of six gram positive bacteria including S. faecalis (Enterobacter faecalis), VRE, Mycobacterium smegmatis (used as an analog and tuberculosis), Bacillus cereus (used as an analog of anthrax) and two strains of streptococci (S. pneumoniae and S. agalacticea), of fabrics treated in accordance with the invention shows significant antimicrobial activity with all four strains.

These results are particularly impressive given that 34% of all nosocomial infections can be attributed to gram positive bacteria (http://www.cdc.gov/ncidod/eid/vol4no3/weinstein.htm).

### BRIEF DESCRIPTION OF THE DRAWINGS

Drawing A is an exploded isometric drawing of one style of a modular unit for treating air to reduce nosocomial infections in clinical settings, using fabrics according to the invention.

Drawing B is an exploded isometric drawing of a second style of modular unit for treating air to reduce nosocomial infections in clinical settings, using fabrics according to the invention, with the unit including a breathing light shelf.

Drawing C is an exploded isometric drawing of a third style of a modular unit for treating air to reduce nosocomial infections in clinical settings, using fabrics according to the invention.

Drawing D is an isometric drawing of apparatus for passively treating air to reduce nosocomial infections in clinical settings, in the form of an upstanding modular vertical fabric array.

Drawing E is an isometric drawing of additional apparatus for passively treating air to reduce nosocomial infections in clinical settings, in the form of an upstanding modular vertical fabric array, similar to that illustrated in Drawing D, in accordance with aspects of the invention.

Drawing F is an isometric drawing of still additional passive apparatus for treating air to reduce nosocomial infections in clinical settings, in the form of an upstanding modular vertical fabric array, similar to that illustrated in Drawings D and E.

Drawing G is a broken isometric drawing of one of the five vertically extending segments of the apparatus illustrated in Drawing D.

Drawing H is a broken isometric drawing of one of the five vertically extending segments of the apparatus illustrated in Drawing F.

Photograph 1 shows test results for a fabric treated in accordance with the method aspects of the invention.

In the drawings, prime and hyphenation notations are used to identify functionally equivalent components incorporated into different embodiments or aspects of the invention, e.g., 14, 14′, 14-1, etc.

### DESCRIPTION OF THE PREFERRED EMBODIMENTS AND BEST MODE KNOWN FOR PRACTICE OF THE INVENTION

Efficacy of the novel fabrics of the invention has been assessed using two standardized methodologies. The first is a modification of the standard American Association of Textile Chemists and Colorists (ATCC) qualitative method 147-1998, termed a “halo” assay (Seshadri and Bhat, 2005). This method involves applying a pure culture of test microbe to cover the surface of a clear nutrient agar plate and overlaying this with small pieces of putative antimicrobial fabric. After a 24 hour incubation period at 37° C., a clear zone of “no growth” is then indicative of antimicrobial activity. An example of the results attained by this method using fabric that has been treated in accordance with the invention, specifically with the eugenol/polyvinyl alcohol/glyoxal preferred embodiment described below, is shown in Photograph 1.

Fabrics in accordance with the invention that show positive for this test have then been further analyzed quantitatively for their ability to reduce microbial growth over a 48 hour period using the standardized ASTM E2149-01 method (Seshadri and Bhat, 2005). This test and verification method is preferable to the standardized ATCC 100 method, as it is suitable for both bacteria and molds as ensuring the optimal contact of the fabric with the suspended microbes (http://www.astm.org/Standards/E2149.htm).

This test and verification method involves addition of 0.5 g of fabric, which have been cut into strips, to a microbial suspension of approximately 1×10⁵ colony forming units (CFU) per ml. After overnight incubation at 37° C. in a shaking bath, the number of viable (living) microbes remaining is determined by performing serial dilutions, further incubating at 37° C. and enumerating visually (Seshadri and Bhat, 2005). Reduction in the numbers of bacteria/fungi are calculated by the following equation, where R=percentage reduction of bacteria/fungi by the specimen treatments, B=number of bacteria/fungi (CFU/ml) recovered from the microbial suspension at the beginning of the experiment and A=number of bacteria/fungi (CFU/ml) recovered from the microbial suspension at the end of the experiment after the 24 hour incubation period (CFU/ml):

$$R = 100 \frac{( B - A )}{B}$$

Each trial was repeated on three separate occasions and the incubations performed in duplicate. This allowed for verification of both microbicidal (killing) fabrics and microbistatic (prevention of multiplication) fabrics; microbicidal fabrics are those where the percentage of viable microbes was reduced by ≧4 log units i.e. growth was decreased up to 10,000-fold or less than 1% of that expected, whereas microbistatic was any decrease in growth of 2 log units or less.

The biocidal actives are successfully coupled to cotton, cotton-polyester and viscose-rayon. Of synthetic fabrics currently available, polyester may be the first choice for commercial value, but is a fairly recalcitrant fabric in terms of accepting color, until heated to around 1000° C. when its pores open and it is able to be dyed.

Cotton-polyester is effective so the percentages of the fabric blend may be adjusted in favor of the polyester to determine a minimum amount of cotton that can be used and still retain biocidal activity.

One aspect of the invention includes affixing natural biocidally active herbal ingredients to fabrics containing 100% cotton, 100% viscose rayon, or 50/50% cotton/polyester. The natural biocidally active ingredients that may be used in practicing the invention include crushed cloves (2% mixed with water to create an aqueous solution), tumeric powder (2% of an aqueous solution), citric acid (5% of an aqueous solution), and corn gluten meal (5% of an aqueous solution). The fabrics (cotton, rayon, cotton/polyester) are immersed in the aqueous solutions for 30 minutes at room temperature and manually stirred at a constant rate. The fabrics are then rinsed in cold water and allowed to dry. Once dry, the fabrics are tested for their antimicrobial activity.

Some aqueous solution treatments do not result in desirably high affixation between the fibers and the herbal ingredient. Different methods of affixing the ingredients to the fiber may be used. Clove oil can be mixed with sodium bicarbonate and applied to the cotton, viscose rayon, and cotton/polyester fabrics. Clove oil has been mixed with acetyl chloride and applied to these three fabrics. Eugenol has been mixed with acetyl chloride and applied to the three fabrics. In each case 5% of the solution was the natural ingredient.

These methods show good results as respecting the bond between the fiber and the herbal ingredient. Combining the natural biocidally active herbal ingredient with polyvinyl alcohol and glyoxal, drying the fabric (that has been soaked with the solution) at an elevated temperature, and then curing the sample at a greater temperature, provides even better bonding of the biocidal herbal to the fabric.

Fabric treatment with 100% cotton based fabrics using eugenol, aloe vera, and copper salt is within the scope of the invention. Greater percentages of the active biocidal herbal ingredients (namely exceeding 5%) do not create a noticeable effect in reduction of microbial activity during testing; lower percentages of the ingredients, (5, 10, or 15 grams of ingredient per liter of aqueous solution) may be used. Eugenol gives, in general, the best results.

Eugenol treatment (with polyvinyl alcohol and glyoxal) of a fabric containing 68% cotton, 30% acrylic, and 2% other fibers is preferable and such fabrics so-treated are preferably used in the apparatus in accordance with the invention. Use of eugenol with polyvinyl alcohol and glyoxal is the preferred way and best mode known for practice of the invention. Eugenol is the preferred biocidally active natural ingredient for use in practicing the invention.

In the procedures described above, all percentages are percentage by weight.

In Drawing A, a unit for treating air to reduce nosocomial infections in clinical settings is shown to be a modular unit designated generally 10 that includes a frame designated generally 12 surrounding an open interior and defining an outer periphery of unit 10. As shown in Drawing A, one or two layers 14 and 14′ of air permeable fabric, treated as described above, are secured about the periphery of frame 12 on a first side 30 of frame 12, with fabric 14 facingly contacting the open interior of frame 12 on first side 30 of frame 12, and with fabric 14′ facingly contacting fabric 14 and lying congruently thereover.

At least one aperture 18 is formed in frame 12. Aperture 18 houses a fan 20 depicted schematically in Drawing A. A second aperture 18′ may also be provided as illustrated to house a second optional fan 20′, or may be used for air bleed.

Fan 20, being housed in aperture 18, serves to blow air from outside of frame 12 into the interior of frame 12 for subsequent passage of substantially all air that is blown into the frame interior, outwardly through fabric 14.

The arrows identified in Drawing A by letters “Ar” indicate the manner of assembly of unit 10, which is shown in Drawing A in a partially exploded isometric view.

The remaining or second side 32 of frame 12 may be open as illustrated, or may be covered with one or more layers of air permeable fabric treated in accordance with the invention.

Still referring to Drawing A, frame 12 has four members, two of which are first and second upstanding lateral members 34 and 36, which are spaced apart as illustrated in Drawing A; the remaining two members of frame 12 are top member 38 and bottom member 40.

Frame 12 further preferably includes first and second diagonal bracing cables 44 and 46, each of which extend from a lower interior corner of frame 12, defined by juncture of bottom 40 and upstanding side member 34 or 36, to a diagonally opposite upper corner, defined by juncture of top 38 with either upstanding side member 36 or upstanding side member 34. Diagonal bracing cables 44 and 46 are secured in place, desirably by connecting with eyes driven into the wood or particle board construction, at a location close to, if not exactly at, the line of juncture between the top and bottom members 22, 24 and the respective side members 34, 36. The eyes and the particular securement of diagonal bracing cables 44 and 46 to frame 12 have not been illustrated to enhance drawing clarity.

The remaining or second side 32 of frame 12 in the unit illustrated in Drawing A has been illustrated open, not covered with fabric. Unit 10 is equipped with a hanging cable 48 connected to second side 32 of frame 12 by suitable screw and collar assemblies, which have not been detailed or numbered in Drawing A to enhance drawing clarity. As shown in Drawing A, screws are driven into the second side 32 of frame 12 at the four corners of second side 32 and collars are then secured in place by screws and permit a small degree of movement of hanging cable 48. Presence of hanging cable 48 facilitates hanging unit 10 on and against a wall, with the wall thereby effectively closing second side 32 of frame 12 if that side is not covered by one or more layers of fabric.

Hanging cable 48 and the unnumbered screws and collars that connect hanging cable 48 to the remainder of the structure may optionally be positioned to maintain frame 12 slightly away from the wall on which unit 10 is mounted. This is desirable when the remaining or second side 32 of frame 12 is covered with one or more layers of air permeable fabric treated in accordance with the foregoing. Unit 10, using hanging cable 48, can be mounted against any reasonably imperforate wall surface; provision of hanging cable 48 permits unit 10 to be mounted essentially flush against the surface of the wall on which unit 10 is mounted. Molly bolts, hooks or the like, driven into a wall may be used to hang unit 10 on the wall.

While unit 10 has been illustrated with two thicknesses of air permeable fabric 14 and 14′, a single fabric thickness may be used, depending on the amount of air moved by fan 20 as selected in specifying fan 20. Additionally, while one or more layers of air permeable fabric, treated in accordance with the invention, may be used on the front and rear surfaces of frame 12, an aesthetically pleasing, air permeable fabric may be used as the outermost fabric 14′ to enhance the aesthetics of unit 10.

Frame 12 of unit 10 is preferably assembled from particle board or wood using adhesive, screws or other mechanical means to secure the parts of frame 12 together in the manner indicated by arrows Ar in Drawing A. The screws, adhesive or other mechanical means used in the assembly of frame 12 have not been illustrated in Drawing A to enhance clarity of the drawing. Frame 12 is preferably of generally rectangular configuration with frame 12 preferably being higher than it is wide.

The air permeable fabric 14 treated in accordance with the foregoing aspects of the invention is preferably secured about the edges of frame 12 that face fabric 14 when fabric 14 and frame 12 are oriented in the position illustrated in Drawing A. Velcro is preferably used to secure fabric 14 to the surfaces of frame 12 that face fabric 14 when those parts are oriented as illustrated in Drawing A. Similarly, Velcro is preferably used to secure fabric 14′ to the surface of fabric 14 when those fabric layers are oriented as illustrated in Drawing A. The Velcro has not been illustrated in order to enhance the drawing. Use of Velcro facilitates replacement of the fabric on a periodic basis.

When unit 10 is assembled by putting the parts of frame 12 in place as indicated by arrows Ar, by positioning fan 20 within aperture 18, and by attaching fabric 14 and 14′ to the facing edges of frame 12 using the preferable Velcro, and unit 10 is either mounted flushly against a wall or has fabric 14′ covering the rear or second side of unit 10, the interior of frame 12 is open other than for the presence of diagonal bracing cables 44, 46. The open construction provides a plenum that is at least partially bounded by fabric 14. When fan 20 operates, fan 20 introduces air into the plenum defined by the interior of unit 10 and forces air gently outwardly through fabric 14 and fabric 14′. Fabrics 14 and 14′ are both air permeable and preferably each treated in accordance with the foregoing in accordance with the invention. Hence, when room air is forced gently into the open interior of unit 10, defining a plenum, and then outwardly through fabric 14, 14′, agents causing nosocomial infections are trapped and killed by fabric 14 and 14′.

As also apparent from Drawing A, frame 12 has a generally rectangular configuration such that first side 30 and second side 32 are parallel one with another and such that top 38 and bottom 40 are parallel one with another. Additionally, the edges, which are unnumbered in the drawings, of the first and second sides 30, 32 and top and bottom 38, 40, are all coplanar, thereby presenting a flat, rectangular, frame-like surface for preferable adhesive securement of the Velcro male or female portion that mates with the counterpart Velcro portion affixed to fabric 14. Fabric 14 and fabric 14′ are both preferably rectangularly shaped and dimensioned to fit congruently with the facing edges of first and second sides 30, 32 and the facing edges of top and bottom 38, 40 defining the rectangular shape of frame 12 so there is no substantial overlap of fabric 14, 14′ respecting frame 12, and so there is no opening between an edge of fabric 14 and a portion of frame 12 through which air could escape without passing through fabric 14.

Referring generally to Drawing B, the apparatus for treating air to reduce nosocomial infections in clinical settings is in the form of a modular unit designated generally 10A that includes a frame designated generally 12A surrounding an open interior and defining an outer periphery of unit 10A. In Drawing B, apparatus 10A is illustrated in a horizontal disposition and, as shown in the left hand portion of Drawing B, is adapted to be used in such a horizontal orientation.

As further illustrated in the left-hand portion of Drawing B, unit 10A is mounted in a horizontal disposition on a unit support frame designated generally 70 positioned within a structure designated generally 60 and in essentially facing contact with the interior surface of a window, or at least the frame of the window, designated generally 58. Unit support frame 70 is maintained in place and vertically supported by cable 68 preferably connected to hooks 66 mounted in the interiorly facing surface of wall 62, above window 58.

Unit support frame 70 preferably includes an inner member designated generally 72 and an outer member designated generally 74 as shown in the left-hand portion of Drawing B. Outer member 74 is dimensioned to vertically support unit 10A by contact with a downwardly facing portion thereof, preferably the downwardly facing portion of frame 12A of unit 10A, as illustrated at the extreme left-hand side of Drawing B. Inner member 72 of unit support frame 70 is dimensioned to receive unit 10A in a facing, complemental manner with unnumbered vertically extending, horizontally facing surfaces of inner member 72 facingly contacting the interiorly positioned one of lateral members 26A and members 22A and 24A. The portion of inner member 72 extending essentially perpendicularly inwardly from window 58 is dimensioned to stop short of the position of fan 20A in aperture 18A, all as illustrated in the extreme left-hand portion of Drawing B.

Optional solar cells 64 may be positioned in facing contact with window 58 to receive sunlight and thereby generate electricity. Solar cells 64 are preferably connected by wires, not shown in the drawings, to fans 20A so that fans 20A are preferably driven by solar energy received through window 58, such that batteries may not be required for fans 20A.

In one preferable implementation illustrated in Drawing B, fabric 14A on the upper side of unit 10A may be a non-woven fabric that is not only air permeable and treated in accordance with the invention, as set forth above, but is also reflective in a manner to reflect natural light, coming in through window 58, throughout the room in which unit 10A is mounted.

Referring specifically to Drawing C of the unit for treating air to reduce nosocomial infections in clinical settings, it is depicted in the form of a modular unit designated generally 10B that includes a frame designated generally 12B surrounding a generally open interior and defining an outer periphery of unit 10B. Referring still to Drawing C, frame 12B and the parts thereof, namely top member 22B, bottom member 24B, lateral members 26B, horizontal interior bracing member 52B, fans 20B and 20B′ and apertures 18B and 18B′ are preferably substantially identical to the correspondingly numbered components of unit 10 illustrated in Drawing A.

In Drawing C, the air permeable fabric that has been treated in accordance with the invention is furnished in the form of modular fabric panels designated generally 54 in Drawing C, where each modular fabric panel includes a frame 56 that is generally of rectangular construction with an open center. Preferably two layers of air permeable fabric 14B and 14B are a part of each modular fabric panel 54 with a first layer of fabric 14B secured to one side of frame 56 and a second layer of fabric 14B′ secured to a second side of frame 56, where the fabric in both instances is preferably secured to frame 56 using Velcro. In Drawing C, to enhance drawing clarity, the frames 56 of modular fabric panels 54 have been illustrated only for modular fabric panels 54 on the right side of the drawing. Similarly, fabric layer 14B has been designated only for those modular fabric panels on the right side of the drawing and fabric layer 14B′ has been designated only for those modular fabric panels on the left side of the drawing.

Each modular fabric panel preferably includes two layers of fabric, one on either side of fabric panel frame 56. Modular fabric panels 54 may be dimensioned such that when mounted on frame 12B there is some overlap of the upper and lower panels by the middle panel as illustrated in Drawing C; unit 10B may also be constructed such that modular fabric panels 54 all collectively fit flushly one against another on one side of frame 12B to present a smooth, continuous surface of air permeable fabric, treated in accordance with the invention as set forth above, for passage of air therethrough.

In one exemplary manifestation, the unit illustrated in Drawing C can be about 19 inches wide and about 44 inches high. As illustrated, three panels of fabric may be positioned on each side of the unit so that there are six (6) fabric panels per unit. Each fabric panel may be about 14 inches by 18 inches and include 2 layers of fabric treated in accordance with the invention. Accordingly, there may be six (6) fabric panels per unit and several such units may be used in a room.

Referring to Drawing D, apparatus for preferably passively treating air to reduce nosocomial infections in clinical settings is depicted in the form of a vertically upstanding array designated generally 100 that includes a frame designated generally 102 for supporting strips of fabric treated in accordance with the foregoing aspects of the invention, where the strips of fabric are designated 14-1, 14-2, 14-3, 14-4 and 14-5. Frame 102 supporting fabric strips 14-1 through 14-5 includes a plurality of upstanding members that are individually designated generally 104. Upstanding members 104 are categorized as first and second upstanding members 106, 108 that are connected front to back by bracing members 110.

Extending laterally between pairs of bracing members 110 and being a part of frame 102 are lateral members 112. In Drawing D, only certain ones of upstanding members 104, first and second upstanding members 106, 108, bracing members 110, and lateral members 112 have been numbered in order to maintain drawing clarity.

Further provided as a portion of frame 102 are cross-braces 114 desirably located at the top of pairs of second upstanding members 108 to increase lateral stability.

A given pair of first and second upstanding members 106, 108 can serve as parts of two adjacent upstanding portions 118 of frame 102 where frame 102 may comprise a number of such adjacent upstanding portions, such as five such portions as illustrated in Drawing D. Two such upstanding portions 118 are indicated and so-designated in Drawing D.

Drawing G illustrates, in vertically truncated form, a broken segment of one of upstanding portions 118. In Drawing G, vertically upstanding members 106 and 108 are positioned at the corners of an imaginary rectangle, where the rectangle is illustrated in dotted lines and designated 120. The one of first upstanding members 106 at the left hand front side of the rectangle 120 is designated 106L in Drawing G, while the one of first upstanding members 106 at the right hand side of rectangle 120 is designated 106R in Drawing G. Similarly, the one of second upstanding members 108 at the left hand side of rectangle 120 is designated 108L in Drawing G and the one of second upstanding members 108 located at the right hand side of rectangle 120 is designated 108R. Upstanding members 106L and 106R are considered to define the front of rectangle 120 where rectangle 120 is provided in this disclosure to clarify the geometry of the structure illustrated in Drawing G.

There may optionally be provided first and second horizontally-oriented support members that are positionable on a floor or other surface to provide vertical support for upstanding portion 118 illustrated in Drawing G; these optional horizontally-oriented support members would run along the respective dotted lines designated 122L and 122R of rectangle 120 in Drawing G.

As further illustrated in Drawing G, a plurality of vertically-spaced apart parallel bracing members 110 connect respective ones of the upstanding first and second members 106, 108 along respective sides of rectangle 120. Bracing members 100 are preferably provided and oriented in closely vertically-spaced, adjacent pairs as illustrated by parallel bracing members 110′, 110″ in Drawing G.

A plurality of lateral members 112 extend between and preferably slideably engage the vertically correspondingly positioned pairs 110′, 110″ of the horizontally-extending parallel bracing members 110. One such lateral member is indicated as 112 in Drawing G. There is further provided a lateral member in the form of a cross-brace 114 at the top of each upstanding portion 118 where the cross-brace 114 is illustrated in Drawing D.

Fabric treated in accordance with the invention as described above, provided in the form of a strip 14-1 as illustrated in Drawing G, is connected at the top of the strip either to an uppermost one of lateral members 112 or to fixed lateral bracing member 114. Fabric strip 14-1 extends downwardly as illustrated in Drawing G and may be positioned in various configurations by adjusting position of lateral members 112 with fabric strip 14-1 passing on a selected side of a given lateral member 112 thereby to provide the desired configuration for fabric strip 14-1. Specifically, lateral members 112 are moveably positionable along the pairs of parallel bracing members 110, between front and rear with respect to rectangle 120, to cause fabric portions 14-1 connected to the lateral members and extending between the lateral members to conform to selected contours. Desirably, a portion of the selected contour, or all of the selected contour, may approximate the upper surface of an air foil, in response to positioning of lateral members 112 and in response to air blowing thereagainst or therealong. Positioning of fabric strip 14-1 as the upper surface of an air foil facilitates generation of vortices along the air foil-like surface, thereby contributing to greater air flow around and along fabric strip 14-1, enhancing the beneficial effects of fabric 14-1.

Optionally, a fixed horizontal brace illustrated as 124 may be provided at the bottom of Drawing G with a fan 126 mounted thereon to blow air upwardly against and along fabric strip 14-1 as indicated by arrows 128 at the top of Drawing G.

Referring to Drawing E, the array 100A shown therein is similar to the array 100 illustrated in Drawing D and is constructed using segments as illustrated in Drawing G. In Drawing E, the upstanding portions 118 illustrated in Drawing G have been horizontally offset one from another front to back, relative to rectangles 120, thereby to provide a different and possibly more efficient configuration for array 100A. Other than the front-to-back offset of upstanding portions 118, array 100A in Drawing E is largely the same as array 100 illustrated in Drawing D, as can be seen by comparing the drawings in which functionally equivalent and substantially corresponding parts have the same number, with the letter “A” used to distinguish parts illustrated in Drawing E from functionally identical or similar corresponding parts in Drawing D.

With respect to array 100A illustrated in Drawing E, a single first upstanding member 106A could not serve as support for adjacent upstanding portions 118A due to the horizontal offset of the upstanding portions 118A as illustrated in Drawing E. However, a first upstanding member 106A of one upstanding portion 118A could serve as a second or rear upstanding member 108A of an adjacent upstanding portion 118A to horizontally offset as illustrated in Drawing E.

Referring to Drawings F and H, Drawing F illustrates another apparatus for passively treating air to reduce nosocomial infections in the form of an array 100B where array 100B includes a frame 102B that has vertically upstanding members 104B positioned at the corners of an imaginary rectangle with one edge of the rectangle being considered the front, in much the same manner as illustrated for Drawings D and G. Further similarly to Drawings D and G, one pair of upstanding members 104B has a first member 106B at the right front of the rectangle and a second member 108B at the right rear of the rectangle, and a second pair of upstanding members 104B having a first member at the left front of the rectangle and second member at the left rear of the rectangle, where the members are designated 106B-L, 106B-R, 108B-L and 108B-R, with these designations being most clearly shown in Drawing H. In array 100B illustrated in Drawing F and in Drawing H, there are further provided a plurality of vertically-spaced apart bracing members 110B connecting respective ones of the upstanding first and second members 106B, 108B of the respective pairs of upstanding members 106B along respective sides of the imaginary rectangle. The imaginary rectangle is not illustrated in Drawing F nor in Drawing G to enhance drawing clarity.

As further illustrated in Drawing F, the fabric treated in accordance with the invention is not provided in the form of vertically elongated strips that extend from the top to the bottom of the apparatus 100B. Rather, the fabric is provided in the form of rectangular sheets 14B where rectangular sheets 14B may be provided as several sheets, one above another, in each upstanding portion 118B of apparatus 100B. Fabric sheets 14B may be secured directly to bracing members 110B desirably by unnumbered rings fitting around bracing members 110B, thereby permitting movement of a fabric sheets 14B between forward upstanding members 106B-L and 106B-R and rear upstanding members 108B-L and 108B-R. Alternatively, lateral members 112B may be provided at either the top or the bottom or both of fabric sheet 14B with lateral members 112B desirably being movable between front and rear along bracing members 110B. With this arrangement, fabric sheets 14B can be adjusted to assume any of a plurality of configurations to take advantage of natural convention in the room in which array 100B is located.

The following is claimed:

1) A method for reducing frequency of nosocomial infections in healthcare institutions comprising the step of providing fabric products for patient contact which fabrics have been treated with a solution of eugenol in sufficient strength and for sufficient time to reduce the percentage of viable microbes in the fabrics by at least 2 log units.

2) A method for treating fabric to impart biocidal properties thereto respecting the bacterium S. aureus and the spore bearing microbe Bacillus cereu, comprising the steps of:
a) preparing an aqueous solution of glyoxal;
b) adding to the solution a natural ingredient that is biocidally active respecting bacterium S. aureus and spore bearing microbe Bacillus cereu and selected from the group consisting of eugenol, cloves, tumeric powder, citric acid, corn gluten meal, aloe vera, and copper salt, in an amount sufficient to reduce the percentage of viable bacterium S. aureus and spore bearing microbe Bacillus cereu in fabric treated with the solution by at least 2 log units;
c) treating the fabric with the solution by:
1) immersing the fabric in the resulting solution;
2) stirring the solution with the fabric therein for about thirty minutes so that the selected biocidally active natural ingredient couples to the fabric;
3) rinsing the fabric with water only; and
4) drying the fabric.

3) The method of claim 2 where in the fabric is selected from fabrics of 100% cotton, 100% viscose rayon, and 50% cotton with 50% polyester.

4) The method of claim 2 wherein the biocidally active natural ingredient is selected from the group consisting of eugenol, cloves, tumeric powder, citric acid, corn gluten meal, aloe vera, and copper salt.

5) The method of claim 2 wherein the solution is aqueous.

6) The method of claim 2 wherein the aqueous solution further comprises polyvinyl alcohol.

7) The method of claim 6 wherein the fabric is 68% cotton, 30% acrylic, and 2% other fibers.

8) The method of claim 2 wherein the biocidally active ingredient is from about 2% to about 5% of the solution, by weight.

9) The method of claim 2 wherein the solution prior to immersion of the fabric therein has 5 grams of naturally biocidally active ingredient per liter of solution.

10) Fabric having a natural biocidally active herbal coupled thereto selected from the group consisting of eugenol, cloves, tumeric powder, citric acid, corn gluten meal, and aloe vera.

11) A method for reducing incidence of nosocomial infections in clinical settings comprising placing a portion of fabric having a natural biocidally active herbal coupled thereto selected from the group consisting of eugenol, cloves, tumeric powder, citric acid, corn gluten meal, and aloe vera, in position in the clinical setting for passive convective air flow along and through the fabric.

12) A method for reducing incidence of nosocomial infections in clinical settings comprising forcing air though fabric having a natural biocidally active herbal coupled thereto selected from the group consisting of eugenol, cloves, tumeric powder, citric acid, corn gluten meal, and aloe vera.

13) Apparatus for reducing incidence of nosocomial infections in clinical settings comprising:
a) fabric having a natural biocidally active herbal coupled thereto selected from the group consisting of eugenol, cloves, tumeric powder, citric acid, corn gluten meal, and aloe vera;
b) a frame having the fabric affixed thereacross; and
c) a fan for blowing air through the fabric affixed across the frame.

14) A garment for wear by workers in clinical settings comprising fabric having a natural biocidally active herbal coupled thereto selected from the group consisting of eugenol, cloves, tumeric powder, citric acid, corn gluten meal, and aloe vera.

15) A method for treating cotton, rayon and cotton-polyester fabrics to impart biocidal properties thereto respecting bacterium S. aureus and spore bearing microbe Bacillus cereu, consisting of only the steps of:
a) preparing an aqueous solution of polyvinyl alcohol and glyoxal;
adding from about 2% by weight to about 5% by
b) weight of eugenol to the solution;
c) immersing the fabric in the resulting solution;
d) stirring the solution with the fabric therein for about thirty minutes at room temperature so that the eugenol couples to the fabric
e) rinsing the fabric with water only; and
f) drying the fabric.

16) A method for treating fabric to impart biocidal properties respecting bacterium S. aureus and spore bearing microbe Bacillus cereu, thereto consisting of only the steps of:
a) preparing an aqueous solution of glyoxal;
b) adding to the solution a natural ingredient that is biocidally active respecting bacterium S. aureus and spore bearing microbe Bacillus cereu selected from the group consisting of eugenol, cloves, tumeric powder, citric acid, corn gluten meal, aloe vera, and copper salt, in an amount sufficient to reduce the percentage of viable bacterium S. aureus and spore bearing microbe Bacillus cereu in fabric treated with the solution by at least 2 log units;
c) treating the fabric with the solution by:
1) immersing the fabric in the resulting solution; and
2) stirring the solution with the fabric therein for about thirty minutes so that the selected natural ingredient that is biocidally active respecting bacterium S. aureus and spore bearing microbe Bacillus cereu couples to the fabric.

17) A method for treating cotton, rayon and cotton-polyester fabrics to impart biocidal properties respecting bacterium S. aureus and spore bearing microbe Bacillus cereu thereto, consisting of:
a) preparing an aqueous solution of glyoxal;
b) adding from about 2% by weight to about 5% by weight of eugenol to the solution;
c) immersing the fabric in the resulting solution;
d) stirring the solution with the fabric therein for about thirty minutes at room temperature so that the eugenol couples to the fabric.

18) The method of claim 17 wherein the aqueous solution includes polyvinyl alcohol.

19) A method for inhibiting growth of the bacterium S. aureus and the spore bearing microbe Bacillus cereu on cotton, rayon and cotton-polyester fabrics by:
a) preparing an aqueous solution of polyvinyl alcohol and glyoxal;
b) adding about 5% by weight of eugenol to the solution;
c) immersing the fabric in the resulting solution;
d) stirring the solution with the fabric therein for about thirty minutes at room temperature to bind the eugenol to the fabric
e) rinsing the fabric with water; and
f) drying the fabric.

20) A method for inhibiting growth of the bacteria S. faecalis, VRE, Mycobacterium smegmatis, Bacillus cereus, S. pneumoniae and S. agalacticea on cotton, rayon and cotton-polyester fabrics by:
a) preparing an aqueous solution of glyoxal;
b) adding about 5% by weight of eugenol to the solution;
c) immersing the fabric in the resulting solution;
d) stirring the solution with the fabric therein for about thirty minutes at room temperature so that glyoxal binds the eugenol to the fabric
e) rinsing the fabric with water; and
f) drying the fabric.

21) A method for treating fabric to impart thereto biocidal properties respecting at least one of the bacteria S. faecalis, VRE, Mycobacterium smegmatis, Bacillus cereus, S. pneumoniae and S. agalacticea by:
a) preparing an aqueous solution of glyoxal;
b) adding to the solution biocidally active eugenol in an amount sufficient to reduce the viable percentage of at least one of the S. faecalis, VRE, Mycobacterium smegmatis, Bacillus cereus, S. pneumoniae and S. agalacticea by at least 2 log units in fabric treated with the solution;
c) treating the fabric with the resulting solution by:
1) immersing the fabric in the resulting solution;
2) stirring the solution with the fabric therein for about thirty minutes so that the biocidally active natural eugenol couples to the fabric;
3) rinsing the fabric with water only; and
4) drying the fabric.

22) A method for treating fabric to impart thereto biocidal properties respecting the bacteria S. faecalis, VRE, Mycobacterium smegmatis, Bacillus cereus, S. pneumoniae and S. agalacticea by:
a) preparing an aqueous solution of glyoxal;
b) adding to the solution about 2 percent by weight of biocidally active natural eugenol to reduce the percentage of viable S. faecalis, VRE, Mycobacterium smegmatis, Bacillus cereus, S. pneumoniae and S. agalacticea bacteria in fabric treated with the solution by at least 2 log units;
c) treating the fabric with the resulting solution by:
1) immersing the fabric in the resulting solution;
2) stirring the solution with the fabric therein for about thirty minutes so that the biocidally active natural eugenol couples to the fabric;
3) rinsing the fabric with water only; and
4) drying the fabric.
